(19) United States
(12) Patent Application Publication
(10) Pub. No.: US 2009/0317900 A1
(43) Pub. Date: Dec. 24, 2009

(54) CHROMATOGRAPHY DEVICE

(75) Inventors: Atsushi HATTORI, Nagoya-shi (JP); Masayoshi MOMIYAMA, Handa-shi (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: AISIN SEIKI KABUSHIKI KAISHA, Kariya-shi (JP)

(21) Appl. No.: 12/480,940

(22) Filed: Jun. 9, 2009

(30) Foreign Application Priority Data
Jun. 18, 2008 (JP) 2008-159437

(51) Int. Cl. C12M 1/00; G01N 30/02

(52) U.S. Cl. 435/287.7; 422/70

(57) ABSTRACT

A chromatography device includes a sample introducing portion for introducing a sample in a liquid state and provided at an upstream side of a flow of the sample, a developing means including a creatinine detecting portion, at which a creatinine detection reagent for detecting creatinine in the sample is provided, and a substance-to-be-detected detecting portion, at which a substance-to-be-detected detecting reagent for detecting a substance included in the sample is provided, and a liquid absorbing portion for absorbing liquid included in the sample in order to allow the sample to flow to a down stream side towards the liquid absorbing portion, wherein the sample introducing portion, the developing means and the water absorbing portion are provided on a board in this order from the upstream side to the downstream side in order to quantify the substance, which is included in the sample and which is to be detected.

### CROSS REFERENCE TO RELATED APPLICATIONS

This application is based on and claims priority under 35 U.S.C. § 119 to Japanese Patent Application 2008-159437, filed on Jun. 18, 2008 the entire content of which is incorporated herein by reference.

### TECHNICAL FIELD

The present invention generally relates to a chromatography device for quantifying a substance, which is included in a sample and which is to be detected.

### BACKGROUND

Creatinine is an end metabolite of a creatine pathway. Generally, creatinine is generated in the muscle fibers of a biological body or is directly generated from phosphocreatine in nerve cells. Alternatively, the creatinine is generated by dehydrating creatine. The creatinine in the muscle fibers or the nerve cells is delivered to the renal glomerulus via the blood, so that the creatinine is excreted in urine. An amount of the creatinine excreted in the urine relative to body weight is substantially consistent for adults. In other words, the creatinine excreted in the urine is proportional to the body weight. Further, the amount of the creatinine excreted in the urine is not influenced by dietary intake or physiological variable factors. The amount of the creatinine excreted in the urine is proportional to a total amount of creatine generated in the muscle fibers. Therefore, concentration level of the creatinine in the urine may be used as an indicator for muscle-related disorders, renal dysfunction or the like.

A test piece for measuring creatinine in biological fluid, which is disclosed in, for example, JP3516002B, includes a supporting body for supporting a developing layer, a reagent layer, which includes 3,4-dinitrobenzoic acid and which is provided on the supporting body, and the developing layer including a strong alkaline substance, which is provided on the reagent layer.

When using the test piece, which is disclosed in JP3516002B, urine and the like, which is used as a sample, is dropped (provided) on the developing layer, and then, changes in color of the reagent layer is measured by using a device, such as a spectral color-difference meter and the like. In order to conduct a quantitative analysis of the sample by using the device, a changing speed in a level of reflection in an absorption wavelength (550 nm) of a condensation product of the creatinine with the 3,4-dinitrobenzoic acid is measured for the sample dropped on the developing layer of the test piece. Then, the concentration level of the creatinine is calculated on the basis of the changing speed by using a standard curve, which is preliminarily obtained on the basis of a result of measuring a creatinine solution of a known concentration.

According to the test piece disclosed in JP3516002B, when the sample is absorbed in the test piece, potential of hydrogen (pH) in the sample, fluid may be maintained at a high level, which may result in stabilizing coloration, which occurs because of a condensation reaction between the creatinine and the 3,4-dinitrobenzoic acid included in the sample. As a result even in a case where a liquid having a strong buffering effect, such as undiluted urine and the like, is used as the sample, the creatinine included in the sample is quantified.

Disclosed in JP2006-349412A is a biosensor, which is obtained by drying and immobilizing a reagente solution, in which creatininase, creatinase, sarcosine oxidase and mediator are soluble in a buffering solution having pH 7 to 8.5, on a board by means of an electrode and the like.

In the biosensor disclosed in JP2006349412A, a buffer material is arranged on the electrode in a solid state together with enzymes such as the creatininase, the creatinase and the sarcosine oxidase. Therefore, a test sample is used as it is without preparing a buffering solution for stabilizing the enzyme when measuring.

Generally, concentration of each component in urine is influenced by dietary intake, water intake, perspiration and the like. Therefore, because the concentration of each component largely fluctuates depending on the level of concentration of the urine, a normal reference value of the sample urine does not exist. Accordingly, in a case where the concentration of target component(s) in the urine is examined, it may be preferable to evaluate the concentration of the target component(s) based on a total amount of urine in a day. However, in a case where a urine test is executed for ambulatory care, it is difficult to collect the urine sample of a day. Hence, generally, a concentration correction of urine (hereinafter referred to as a creatinine correction) is executed by using a ratio obtained by dividing a creatinine value in the urine sample by a preliminarily obtained creatinine value of known concentration.

As is mentioned above, the creatinine, which is included in the urine, is not influenced by physiologically variable factors. Therefore, a total amount of the creatinine excreted in the urine sample in a day is proportional to a total amount of the creatinine created in the muscle fiber. For example, when assuming that the total amount of the creatinine excreted in a day is one gram (1 g), the total amount of the urine in a day may be estimated by executing the creatinine correction, on the basis of the obtained concentration of the creatinine in the sample urine.

According to the known methods, in order to obtain a value used for executing the creatinine correction, the creatinine needs to be measured differently from the component measurement of the urine, which is used as the sample. Therefore, an additional process needs to be executed to the sample urine in the known component measurement.

A need thus exists to provide a chromatography device which is not susceptible to the drawback mentioned above.

### SUMMARY OF THE INVENTION

According to an aspect of the present invention, a chromatography device includes a sample introducing portion for introducing a sample in a liquid state and provided at an upstream side of a flow of the sample, a developing means including a creatinine detecting portion, at which a creatinine detection reagent for detecting creatinine in the sample is provided, and a substance-to-be-detected detecting portion, at which a substance-to-be-detected detecting reagent for detecting a substance included in the sample and is to be detected is provided, and a liquid absorbing portion for absorbing liquid included in the sample in order to allow the sample to flow to a down stream side towards the water absorbing portion, wherein the sample introducing portion, the developing means and the water absorbing portion are provided on a board in this order from the upstream side to the downstream side in order to quantify the substance, which is included in the sample and which is to be detected.

### BRIEF DESCRIPTION OF THE DRAWINGS

The foregoing and additional features and characteristics of the present invention will become more apparent from the following detailed description considered with the reference to the accompanying drawings, wherein:

FIG. 1A is a diagram schematically illustrating a chromatography device when being viewed from a side thereof;

FIG. 1B is a diagram schematically illustrating the chromatography device when being viewed from above;

FIG. 2 is a graph illustrating a relationship between color density and creatinine concentration;

FIG. 3 is a diagram, illustrating a standard curve, which is obtained on the basis of quantification results of IL-6 concentration; and

FIG. 4 is a diagram illustrating a relationship between creatinine correction value, which is calculated on the basis of the standard curve, and creatinine correction value (a comparative value), which is calculated on the basis of a known concentration.

### DETAILED DESCRIPTION

An embodiment of a chromatography device will be described below in accordance with the attached drawings.

Various examination methods are used for clinical examinations. For example, a dry chemistry method is known as one of the examination methods. In the dry chemistry method, a sample in a liquid state is dropped (provided) on a reagent, which is provided on a developing matrix, such as a film, a test piece and the like, in a dry state, in order to measure a substance, which is included in the sample and which is to be examined. Accordingly, the dry chemistry method does not need preparation for enzymatic reaction liquid or piping system, which are required for a liquid method. Therefore, the dry chemistry method is easily operated and maintained.

The chromatography device is, for example, an immunochromatography, having the reagent on a developing matrix (a developing means) with a single layer. In the chromatography device, a substance to be detected is separated from other substances in the sample by a mobile phase (e.g. liquid and the like) including the substance to be detected passing on a surface or through an inner side of the developing means, which is a stationary phase. Accordingly, the substance, which is included in the sample and which is to be examined, is quantified. In this embodiment, a urine sample including creatinine is used as the sample.

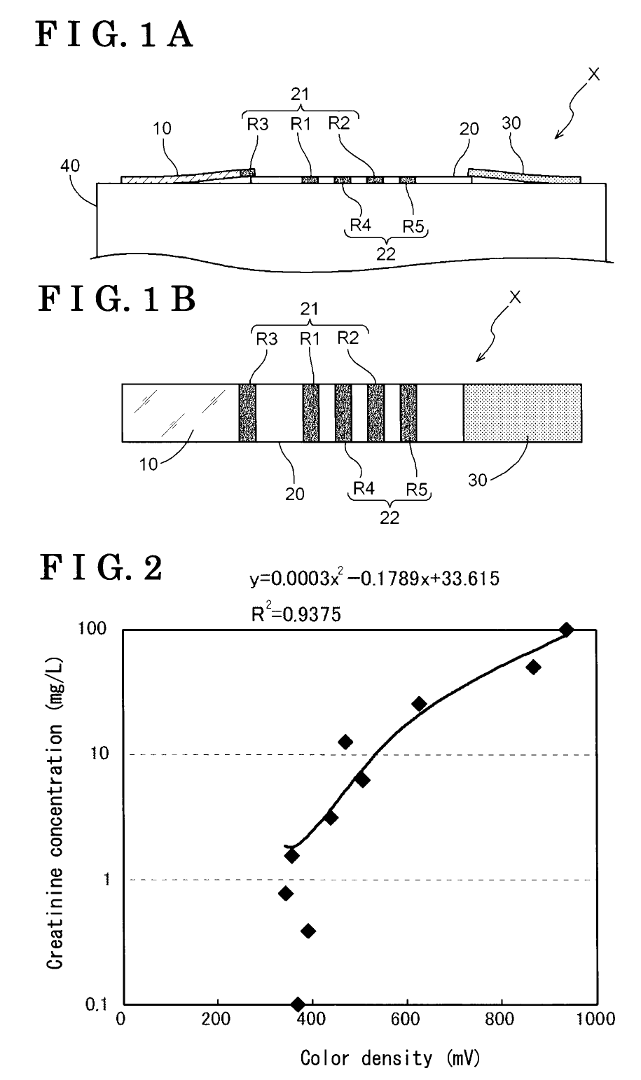

As illustrated in FIG. 1 (FIGS. 1A and 1B), in a chromatography device X, a sample introducing portion 10 for introducing the sample, a creatinine detecting portion 21 for supporting a creatinine detecting reagent detecting the creatinine, a developing means 20 including a substance-to-be-detected detecting portion 22 for supporting a substance-to-be detected detecting reagent detecting the substance, which is included in the sample and which is to be detected, and a liquid (water) absorbing portion 30 are provided on a board 40 in this order from left to right in FIG. 1.

### <Board>

Any desired shape, size and material may be used for the board 40 as long as the sample introducing portion 10, the developing means 20 and the liquid absorbing portion 30 are provided thereon. However, materials, which do not inhibit liquid (water) absorbability of the liquid absorbing portion 30, need to be used to the board 40. For example, a plastic sheet in a thin plate shape, may be used as the board 40 in order to facilitate carriage of the chromatography device. For example, in this embodiment, the board 40 is formed in a size of approximately 60 mm*5 mm*0.3 mm.

<Sample Introducing Portion>

The sample introducing portion 10 is a portion on which the sample is dropped (provided). Further, the sample introducing portion 10 is provided at a upstream side of flow of the sample at the chromatography device X. In this embodiment, for example, a glass filter (Millipore Corporation) is used as the sample introducing portion 10. However, the sample introducing portion 10 is not limited to the glass filter, but any desired material may be used as long as that selected material allows the mobile phase to flow in a downstream side of the flow of the sample by the capillarity.

<Developing Means>

In the developing means 20, the creatinine detecting portion 21 for supporting the creatinine detecting reagent detecting the creatinine and the substance-to-be-detected detecting portion 22 for supporting the substance-to-be-detected detecting reagent detecting the substance to be detected are provided on a carrier of the stationary phase. An immunochromatographic test is one of examination methods using an antigen-antibody reaction and the capillarity. Therefore, for example, a nitrocellulose membrane (Millipore Corporation) and the like, which allows the mobile phase to flow towards the downstream side by the capillarity, may be used as the developing means 20. The developing means 20 is arranged so that one end portion of the developing means 20 is arranged so as to contact or overlap with a downstream side portion of the sample introducing portion 10 and so that the other end portion of the developing means 20 is arranged so as to contact or overlap with an upstream side portion of the water absorbing portion 30.

When quantifying the substance to be examined, the sample including the substance to be detected (antigen) is provided at the sample introducing portion 10 in order to develop the sample to the developing means 20 by the capillarity. Then, the antigen is identified, existence of the antigen is determined or an amount of the antigen is measured by coloring a reactive portion (by changing color density of the reactive portion) by using the antigen-antibody reaction in a sandwich-type (a sandwich method). The reactive form of the antigen-antibody is not limited to the sandwich method, but a competitive antigen-antibody reaction (a competition method) may be used. For example, the sandwich method may be used in a case where a molecular mass of the substance to be detected is great. On the other hand, the competition method may be used in a case where the molecular mass of the substance to be detected is low.

According to the chromatography device X, because the chromatography device X includes the creatinine detecting portion 21 and the substance-to-be-detected detecting portion 22, the creatinine included in the sample and the component of the sample are simultaneously measured. Accordingly, the measurement of the creatinine included in the sample and the component of the sample are easily measured without separately measuring the creatinine and the component of the sample. Therefore, a value for a concentration correction of sample executed for a creatinine concentration is easily obtained (hereinafter, the correction is referred to as a creatinine correction, and the value is referred to as a creatinine correction value).

The creatinine detecting portion 21 includes a first reagent portion R1 including creatininase, creatinase and sarcosine oxidase, and a second reagent portion R2 including peroxidase. Further, a detection reagent portion R3 including a chromogenic substrate is provided at either the sample introducing portion 10 or at the developing means 20. In this embodiment, the detection reagent portion R3 is provided in the vicinity of an end portion of the sample introducing portion 10 at the downstream side. Reagents are supported on the first reagent portion R1, the second reagent portion R2 and the detection reagent portion R3, respectively, in the dry state. In this embodiment, the detection reagent portion R3 is provided at the creatinine detection portion 21 at the upstream side relative to the second reagent portion R2.

In this embodiment, 3,3′-Diaminobenzidine tetrahydrochloride (DAB) is used as the chromogenic substrate included in the detection reagent portion R3. However, the chromogenic substrate is not limited to the DAB. For example, a colloid substance such as gold colloid, latex and the like, quantum dot, fluorochrome such as fluorescein isothiocyanate (FITC) and the like, and an enzyme substance such as alkaline phosphatase, galactosidase and the like may be used as the chromogenic substrate.

As long as a concentration of each of the enzymes included in the first reagent portion R1 fall within a predetermined concentration range (e.g. the concentration of the creatininase is in a range of 0.1 to 100 U/μL, the concentration of the creatinase is in a range of 0.1 to 100 U/μL, and the concentration of the sarcosine oxidase is in a range of 0.1 to 100 U/μL, preferably, the concentration of the creatininase is in a range of 0.1 to 10 U/μL, the concentration of the creatinase is in a range of 0.1, to 10 U/μL, the concentration of the sarcosine oxidase is in a range of 0.1 to 1 U/μg), the creatinine included in the sample is promptly quantified without providing the enzymes excessively on the chromatography device. Therefore, costs for enzymes are reduced. The concentration ranges of the enzymes are appropriately set in view of a material used for the developing means 20, a speed of flow of the mobile phase and the like. For example, appropriate concentration ranges that allow the detection reagent to be sufficiently colored are set, depending on whether the developing means 20 is configured by a material that increases the speed of flow of the mobile phase or whether the developing means 20 is configured by a material that reduces the speed of flow of the mobile phase.

After applying (dropping) the sample on the sample introducing portion 10, the creatinine included in the urine sample passes through the detection reagent portion R3, the first reagent portion R1 and the second reagent portion R2 with the mobile phase flowing towards the downstream. While the creatinine included in the urine sample passes through the detection reagent portion R3, the first reagent portion R1 and the second reagent portion R2, the following series of reactions occur. The creatinine generates creatine by the enzymatic reaction with the creatininase. The generated creatine generates sarcosine in response to the enzymatic reaction with the creatinase. The sarcosine generates glycine and hydrogen peroxide in response to the enzymatic reaction with the sarcosine oxidase. The generated hydrogen peroxide acts on the peroxidase, thereby coloring the DAB (for example, the DAB is colored and then a color density of the DAB changes). In the above-described series of reactions, the hydrogen peroxide is generated in response to the amount of the creatinine. Therefore, the DAB is colored (for example, the color density of the DAB is changed) in response to the generated amount of the hydrogen peroxide.

By providing the detection reagent portion R3 at the upstream side relative to the second reagent portion R2, as described above, the chromogenic substrate included in the detection reagent portion R3 flows towards the downstream side together with the creatinine included in the sample. Therefore, when the substance (the hydrogen peroxide), which is generated as a result of the enzymatic reaction induced when the sample passes through the creatinine detecting portion 21, acts on the peroxidase, the chromogenic substrate is quickly colored. Accordingly, the detection reagent portion R3 is provided either at the downstream side or the upstream side of the chromatography device.

A first antibody and a second antibody, which are immobilized, are provided at the substance-to-be-detected detecting portion 22 at different positions. More specifically, in this embodiment, the first antibody and the second antibody are supported at a first capture portion R4 and a second capture portion R5, respectively, in the dry state. As long as the first capture portion R4 and the second capture portion are provided to the chromatography device X in this order from the upstream to the downstream, the first and second capture portions R4 and R5 may be provided at the chromatography device X in the upstream side relative to the first and second reagent portions R1 and R2 of the creatinine detecting portion 21.

A substance, which reacts with immune antibody, a capture agent of the immune antibody, or urinary constituent may be provided at the developing means 20, in order to determine whether or not the measurement is completed or whether or not a specific constituent is included in the sample.

<Liquid Absorbing Portion>

The liquid absorbing portion 30 absorbs the liquid, which is the mobile phase existing at the neighboring developing means 20. When the liquid absorbing portion 30 absorbs the liquid at the developing means 20, the sample, which is provided at the sample introducing portion 10, flows to the downstream side towards the liquid absorbing portion 30. In this embodiment, a filter paper (Millipore Corporation), which serves as the liquid absorbing portion 30 and which is cut in an appropriate size, is arranged at the chromatography device so as to contact or overlap with the a downstream side portion of the developing means 20.

<Sample>

The sample, used in the embodiments, refers to a fluid sample including or supposed to include the substance(s), which is to be detected and which is to be an object to be quantified. Fluid based on any derivations may be used as a sample. However, in this embodiment, a sample, which includes the creatinine and whose concentration is corrected on the basis of the creatinine concentration, such as the urine obtained from a biopsy specimen and the like, is used as an example.

The substance, which is included in the sample and which is to be detected, is captured by being combined with a binding substance, which may generate a specific combination (a specific complex) together with the substance to be detected. The specific complex is generated as a result of execution of an assay using a binding pair. As described above, an immunochemical complex, which is generated as a result of the antigen-antibody reaction, a complex, which is generated as a result of hybridization between complementary nucleic acids, and the like is used as an example of the specific complex. Any substances, such as a micro-molecule, a deoxyribonucleic acid fragment (DNA fragment) and the like of, for example, a chemical substance, protein and the like, an interleukin, an microorganism, a virus, a fragment, a hormone, a lipid-derived excrete infinitesimal substance of the microorganism, the virus and the like, are used as a target.

[First Examination]

<Quantification of Creatinine>

In order to quantify the creatinine included in the sample by using the chromatography device X, firstly, the following reagents are provided at the creatinine detecting portion 21 (the first reagent portion R1 and the second reagent portion R2). In the first examination, the reagent is not provided at the substance-to-be-detected detecting portion 22. A reagent, which is created by mixing the creatininase (Kikkoman Corporation) at 10 U/μL, the creatinase (Kikkoman Corporation) at 10 U/μL and the sarcosine oxidase (Toyobo Co., Ltd.) at 1 U/μL, is applied and dried on the first reagent portion R1. The peroxidase (Nacalai Tesque, Inc.) at 100 μg/μL is applied and dried on the second reagent portion R2. The 3, 3′-Dimethylaminoazobenzene (DAB) (Dojindo Laboratories) at 1 mg/ml is applied and dried on the detection reagent portion R3 of the sample introducing portion 10.

Artificial urine is produced by preparing reagents indicated in Table 1. The artificial urine does not include the creatinine at this point.

TABLE 1

| Artificial Urine (pH 6.4) | (g/L) |
|---|---|
| Urea | 16 |
| Uric acid | 0.63 |
| Citric acid | 0.54 |
| Na₂SO₄ | 4.31 |
| KCl | 3.43 |
| NaH₂PO₄ | 2.73 |
| NaCl₂ | 2.32 |
| NH₄Cl | 1.06 |
| CaCl₂/2H₂O | 0.63 |
| MgCl₂ | 0.45 |

The creatinine (Wako Pure Chemical Industries, Ltd.) is added to the artificial urine including the components indicated in Table 1. Then, a colored amount of the reagent is measured by using a known optical colored amount measuring device. Various concentration levels are set for the creatinine in a range between 0.1 to 100 mg/L. Then, the colored amount (coloring reaction) of the detection reagent (i.e. coloring (changes in a color density) of the detection reagent occurring when the detection reagent reacts against the creatinine) in each concentration is measured. Specifically, voltage values, which are obtained by detecting light absorption of the sample in each concentration by means of a photodiode, are determined as color density (mV). A standard curve is plotted on the basis of the measurement result (see FIG. 2). Further, as is evident from the result indicated in FIG. 2, it is proven that the creatinine is properly measured by using the chromatography device X.

[Second Examination]

<Quantification of Interleukin 6 (IL-6)>

Interleukin 6 (IL-6) is quantified by using the chromatography device X. More specifically, the IL-6 is quantified by using the antigen-antibody reaction by the sandwich method. The following reagents are provided at the substance-to-be-detected detecting portion 22 at the developing means 20 of the chromatography device X having the configuration described above. The first capture portion R4 is provided at the upstream side relative to the second reagent portion R2 as the substance-to-be-detected detecting portion 22. Further, the second capture portion R5 is provided at the downstream side relative to the second reagent portion R2 as the substance-to-be-detected detecting portion 22. Rabbit anti-human interleukin 6 antibody (Santa Cruz Biotechnology, Inc.) at a concentration of 200 μg/mL is applied and dried on the first capture portion R4. Further, anti-rabbit IgG antibody (Sigma Aldrich, Inc.) at a concentration of 1 mg/mL is applied and dried on the second capture portion R5.

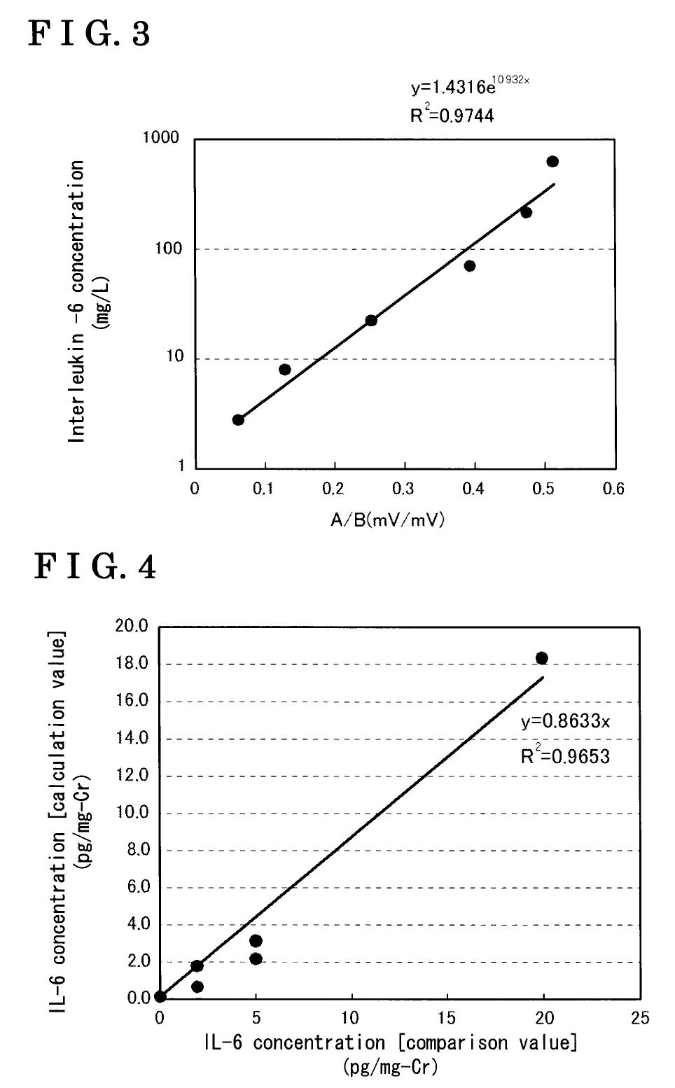

A sample, which is created by mixing 5 μL of the rabbit anti-human interleukin-6 (Santa Cruz Biotechnology, Inc.) at a concentration of 200 μg/mL with 1 mL of the artificial urine, which is created by the components used in the first examination, and further, adding human interleukin-6 (human IL-6) (Pepro Tech Ec, Inc.), is provided at the sample introducing portion 10 of the chromatography device X. Then, alkaline phosphatase-conjugated goat anti-rabbit IgG antibody (Kirkegaard & Perry Laboratories, Inc.), which is adjusted to a concentration of 1 μg/mL by means of a phosphoric acid-buffering solution (pH 9.5), is provided at the sample introducing portion 10. Furthermore, an appropriate amount of 6-Bromo-4-chloro-3-indolyl phosphate/nitro blue tetrazolium chloride (BCIP/NBT) (Nacalai Tesque, Inc.) is provided at the sample introducing portion 10. Then, the colored amount of the sample in each concentration is measured by the colored amount measuring device. Accordingly, a standard curve is plotted on the basis of the measurement result (see FIG. 3).

[Third Examination]

<IL-6 Obtained after Creatinine Correction>

The creatinine, which has a concentration level raging between 5 to 50 mg/L, is mixed to the artificial urine having the components described in the first examination. Further, the IL-6, which has a concentration level raging between 10 to 100 μg/L, is mixed to the obtained compound liquid (Sample 1, 2, 3, 4, 5, and 6). The creatinine concentration and IL-6 concentration of each sample is indicated in Table 2.

TABLE 2

| | Sample Number | | | | | |
|---|---|---|---|---|---|---|
| | 1 | 2 | 3 | 4 | 5 | 6 |
| Creatinine | 5 | 5 | 10 | 10 | 50 | 50 |
| Concentration (mg/L) | | | | | | |
| IL-6 Concentration | 10 | 100 | 50 | 50 | 100 | 10 |
| (pg/L) | | | | | | |

5 μL of the rabbit anti-human interleukin-6 antibody (Santa Cruz Biotechnology, Inc.) at the concentration of 200 μg/mL is mixed with 1 ml of each sample, and then, the compound sample is provided to the sample introducing portion 10 of the chromatography device X, which has a configuration described in the second examination, in order to cause a reaction. Then, the alkaline phosphatase-conjugated goat anti-rabbit IgG antibody, which is prepared to have the concentration of 1 μg/mL by the phosphoric acid-buffering solution (pH 9.5), is provided at the sample introducing portion 10, Furthermore, an appropriate amount of the BCIP/NBT (Nacalai Tesque, Inc.) is provided at the sample introducing portion 10 and the colored amount of the sample in each concentration is measured by the colored amount measuring device. The result of measurement is indicated in Table 3. Symbols “A/B (mV/mV)” indicates “a dimensionless quantity obtained by dividing a voltage value at the second capture portion R5 by a voltage value at the first capture portion R4”.

TABLE 3

| | Sample Number | | | | | |
|---|---|---|---|---|---|---|
| | 1 | 2 | 3 | 4 | 5 | 6 |
| Color density | 372 | 302 | 449 | 520 | 688 | 673 |
| (mV) | | | | | | |
| A/B (mV/mV) | 0.12 | 0.41 | 0.31 | 0.32 | 0.38 | 0.15 |

The creatinine concentration and the IL-6 concentration are calculated on the basis of the standard curves illustrated in FIGS. 2 and 3. The calculation results are indicated in Table 4.

TABLE 4

| | Sample Number | | | | | |
|---|---|---|---|---|---|---|
| | 1 | 2 | 3 | 4 | 5 | 6 |
| Creatinine concentration | 8.6 | 6.9 | 13.8 | 21.7 | 52.5 | 49.1 |
| (mg/L) | | | | | | |
| IL-6 concentration (pg/L) | 5.3 | 126.6 | 42.4 | 47.3 | 91.2 | 7.4 |

The IL-6 concentration, to which the creatinine correction is executed, is calculated on the basis of the results indicated in Table 4. The calculation results of the IL-6 concentration, which is creatinine-corrected, is indicated in Table 5. For example, the creatinine-corrected IL-6 concentration in the case of the sample 1 is calculated to 0.6 (pg/mg) (5.3/8.6=0.6 (pg/mg)). In the third examination, the concentration of the creatinine and the concentration of the IL-6 are preliminarily obtained. Therefore, the value, to which the creatinine correction is preliminarily executed, may be calculated. Therefore, the creatinine correction values, which are preliminarily calculated, are provided at the bottom-most row in Table 5 as comparative data.

TABLE 5

| | Sample Number | | | | | |
|---|---|---|---|---|---|---|
| | 1 | 2 | 3 | 4 | 5 | 6 |
| Creatinine-corrected IL-6 | 0.6 | 18.2 | 3.1 | 2.2 | 1.7 | 0.2 |
| concentration (pg/mg) | | | | | | |
| (Comparative Value) | 2 | 20 | 5 | 5 | 2 | 0.2 |
| Creatinine-corrected IL-6 | | | | | | |
| concentration (pg/mg) | | | | | | |

In order to verify the results indicated in Table 5, the values, which are obtained after the creatinine correction is executed, are graphed (see FIG. 4). As a result, it may be determined that the creatinine correction value, which is calculated on the basis of the standard curve, correlates with the creatinine correction value (the comparative value), which is calculated on the basis of a sample having a known concentration. Accordingly, the creatinine correction value, which is calculated by using the chromatography device X, is determined to be valid.

Chromatography, which serves as the chromatography device X, separates the substance to be detected from other substances included in the sample by the mobile phase (e.g. liquid and the like) including the substance to be detected passing on the surface or through the inner side of the developing means 20, which is a stationary phase. Accordingly, in the chromatography device X, the substance to be detected is quantified by separating and purifying the substance to be detected in the sample.

According to the chromatography device X of the embodiment, the creatinine detecting portion 21 and the substance-to-be-detected detecting portion 22 are provided at the developing means 20. Accordingly, the creatinine is measured and, at the same time, the substance to be detected is quantified. Therefore, the measurement of the creatinine and the substance to be detected is easily performed. Furthermore, the creatinine correction value is easily obtained. Moreover, according to the chromatography device X of the embodiment, because the reagent is preliminarily provided at the developing means 20, preparation of the reagent for quantification of the sample is not necessary, which may result in performing the examination promptly. Furthermore, the chromatography device X of the embodiment may enhance portability and storage. Additionally, the chromatography device X of the embodiments is easily adaptable to a point-of-care testing, which is a diagnostic testing given near a site of patient.

According to the embodiment, the creatinine detecting portion 21 includes the first reagent portion R1, having creatininase, creatinase and sarcosine oxidase, and the second reagent portion R2 having peroxidase. The detection reagent portion R3 including a chromogenic substrate is provided either at the sample introducing portion 10 or at the developing means 20. Further, the detection reagent portion R3 is provided at the upstream side relative to the second reagent portion R2.

After the sample is provided at the sample introducing portion 10, the creatinine, which is included in the urine sample, passes through the detection reagent portion R3, the first reagent portion R1 and the second reagent portion R2, as the mobile phase flows towards the downstream side towards the liquid absorbing portion 30. When the creatinine passes through the detection reagent portion R3, the first reagent portion R1 and the second reagent portion R2, the following series of reactions occur. The creatinine generates the creatine by the enzymatic reaction with the creatininase. The generated creatine generates the sarcosine in response to the enzymatic reaction with the creatinase. The sarcosine generates the glycine and the hydrogen peroxide in response to the enzymatic reaction with the sarcosine oxidase. The generated hydrogen peroxide acts on the peroxidase, thereby coloring the chromogenic substrate.

Accordingly, by providing the detection reagent portion R3 at the upstream side relative to the second reagent portion R2, the chromogenic substance included in the detection reagent portion R3 flows towards the downstream side together with the creatinine included in the sample. Therefore, when the substance, which is generated as a result of the enzymatic reaction induced when the sample passes through the creatinine detecting portion 21, acts on the peroxidase, the chromogenic substrate is quickly colored.

According to the embodiment, the concentration of the creatininase is set in the range between 0.1 to 100 U/μL, the concentration of the creatinase is set in the range between 0.1 to 100 U/μL and the concentration of the sarcosine oxidase is set in the range between 0.1 to 100 U/μL.

Accordingly, the creatinine included in the sample is promptly quantified without providing the enzymes excessively on the chromatography device X. Therefore, costs for enzymes are reduced.

According to the embodiment, the first reagent portion R1 is provided at the upstream side relative to the second reagent portion R2.

The principles, preferred embodiment and mode of operation of the present invention have been described in the foregoing specification. However, the invention which is intended to be protected is not to be construed as limited to the particular embodiments disclosed. Further, the embodiments described herein are to be regarded as illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

1. A chromatography device, comprising:
a sample introducing portion for introducing a sample in a liquid state and provided at an upstream side of a flow of the sample;
a developing means including a creatinine detecting portion, at which a creatinine detection reagent for detecting creatinine in the sample is provided, and a substance-to-be-detected detecting portion, at which a substance-to-be-detected detecting reagent for detecting a substance included in the sample and is to be detected is provided; and
a liquid absorbing portion for absorbing liquid included in the sample in order to allow the sample to flow to a down stream side towards the liquid absorbing portion, wherein
the sample introducing portion, the developing means and the liquid absorbing portion are provided on a board in this order from the upstream side to the downstream side in order to quantify the substance, which is included in the sample and which is to be detected.

2. The chromatography device according to claim 1, wherein the creatinine detecting portion includes a first reagent portion, having creatininase, creatinase and sarcosine oxidase, and a second reagent portion having peroxidase, a detection reagent portion including a chromogenic substrate is provided either at the sample introducing portion or at the developing means, and wherein the detection reagent portion is provided at the upstream side relative to the second reagent portion.

3. The chromatography device according to claim 1, wherein a urine sample is used as the sample.

4. The chromatography device according to claim 2, wherein concentration of the creatininase is set in a range between 0.1 to 100 U/μL, concentration of the creatinase is set in a range between 0.1 to 100 U/μL and concentration of the sarcosine oxidase is set in a range between 0.1 to 100 U/μg.

5. The chromatography device according to claim 3, wherein the first reagent portion is provided at the upstream side relative to the second reagent portion.
